(19) United States
(12) Patent Application Publication
(10) Pub. No.: US 2023/0265415 A1
(43) Pub. Date: Aug. 24, 2023

(54) GENERATION OF DIVERSE VIRAL LIBRARIES

(71) Applicant: THEOLYTICS LTD., Oxford (GB)

(72) Inventor: Margaret DUFFY, Oxford (GB)

(21) Appl. No.: 18/011,999

(22) PCT Filed: Jun. 24, 2021
(86) PCT No.: PCT/GB2021/051609

§ 371 Date: Dec. 21, 2022

(30) Foreign Application Priority Data
Jun. 25, 2020 (GB) 2009701.0

(51) Int. Cl. C12N 15/10; C12N 7/00

(52) U.S. Cl. CPC C12N 15/1058; C12N 7/00; C12N 2710/10321; C12N 2710/10351; C12N 2710/10332

(57) ABSTRACT

This invention relates to a process for producing a library of viruses, comprising first and second culturing steps. These steps aim to promote intra-species and inter-species recombination, respectively, between double-stranded DNA viruses of the same virus family.

This invention relates to a process for producing a library of viruses, comprising first and second culturing steps. These steps aim to promote intra-species and inter-species recombination, respectively, between double-stranded DNA viruses of the same virus family.

Viruses have huge potential for use in a wide range of applications such as oncolytic viruses, vaccines, anti-viral drugs and gene-therapy vectors. To date, the majority of methods for generating viruses with desired properties have depended on incremental understandings of the intricacies of any particular viral genome. Modifications to the virus genomic sequence are then designed in order to introduce the desired characteristics into the genome, with the hope that this will render that virus suitable for a specific application.

A new approach is needed to speed up the process of generating viruses with diverse properties in order to provide a library of high diversity from which the best virus with the most desired properties for a specific application can be selected.

One example of the use of a diverse viral library is in the development of new oncolytic vectors. There are significant ongoing challenges in developing treatments for cancers, due to the ability of tumours to acquire resistance to treatment. Current therapies, including chemotherapy and radiotherapy, are associated with toxic side-effects and are often of limited efficacy. In order to overcome these issues, oncolytic viruses have been used as a new approach. Naturally-occurring or genetically-modified oncolytic viruses have the ability to selectively-replicate within tumours, infect and kill cancer cells via cell lysis without affecting normal cells, and trigger anti-tumour immune response from the release of tumour antigens. To ensure that oncolytic viruses do not replicate in normal cells, non-virulent strains are selected or the viral genome is genetically-engineered either by inserting or deleting genes. Examples of genetically-engineered oncolytic viruses include Oncorine (H101), an adenovirus with a deletion in the E1B55k, which was approved in China for use in head and neck cancers; and T-Vec, a herpes virus with deletions in the γ34.5 and α47 genes, which was approved by the FDA for use in melanomas. However, genetically-engineered oncolytic viruses to date have had limited success in the clinics.

Although, there are >60 serotypes of wild-type human adenoviruses (Ad), Ad5 and variants thereof are the dominant Ad types used as oncolytic adenoviruses. The vast majority of other serotypes have not been explored for use as oncolytic viruses.

One approach for generating further oncolytic viruses is to use bioselection or ‘directed evolution’ using viral pools to generate new oncolytic viral serotypes. This involves mimicking natural selection of viruses by pooling different viral serotypes and/or randomly introducing mutations (described for example in Bauzon and Hermiston, “Oncolytic viruses: The Power of Directed Evolution”; Advances in Virology, vol. 2012, Article ID 586389). Kuhn et al. showed the use of directed evolution to generate a novel non-Ad5 oncolytic virus for the treatment of colon cancer (“Directed Evolution generates a novel oncolytic virus for the treatment of colon cancer”; PLoS One 3(6): e2409 (2008); and WO2008/080003) and ovarian cancer (“OvAd1, a Novel, Potent, and Selective Chimeric Oncolytic Virus Developed for Ovarian Cancer by 3D-Directed Evolution” Mol. Ther. Oncolytics; 4: 55-66). Kuhn and Hermiston et al. pooled different Ad subgroups (Ad3, Ad4, Ad5, Ad9, Ad11p, Ad16, Ad35, Ad40) with or without the chimeric virus ColoAd1. Random mutations were introduced to a sub-sample of the viral pool and then added to the non-mutated pool of viral particles. The combined viral pool was passaged on human tumour cell lines to invite recombination to increase diversity. This resulted in the generation of ColoAd1 (Ad3/Ad11p) and OvAd1 (Ad3/ColoAd1) chimeric oncolytic viruses with increased potency and a larger therapeutic window in comparison to Ad5. As a result of the ‘directed evolution’ approach, the genome of ColoAd1 was modified, including deletions in the E3 and E4 regions, alterations in the E2B region and deletion that maps to the E4orf4 region of the virus. The increased potency associated with the chimeric virus could be explained by the changes in the viral genome. In another example, described in WO2008/080003, a method of introducing point mutations using nitrous acid to obtain approximately 10 mutations per viral genome followed by recombination of the viral pools is described.

There is still a need for improved methods for increasing the diversity of a viral pool to provide a starting point for screening/bioselection for improved, cell-specific oncolytic viruses.

In Kuhn et al. (2008, supra), 3 serotypes from Ad group B and 1 serotype from each of Ad groups C, D, E and F were pooled together and passaged on cultures of target tumour cell lines under conditions which were said to invite recombination between serotypes. The result was ColoAd1, a complex Ad3/Ad11p chimeric virus. Ad3 and Ad11p are both Ad group B serotypes, and hence the recombination in ColoAd1 resulted from an intra-group (AdB) recombination, i.e. the viruses from Ad groups C, D, E and F were not involved.

In a later paper by Kuhn et al. (Molecular Therapy: Oncolytics, vol. 4, March 2017, pp. 55-66), a further chimeric Ad virus was produced, OvAd1. This was the result of a recombination between Ad3, Ad11 and ColoAd1, all of which were AdB serotypes or chimeras.

The inventors have now recognised, however, that different adenovirus species grow at different rates on different cell lines and that different cell lines have different propensities to allow viral recombination. Hence it may not be possible to promote the best recombination diversity in a mixed set of adenovirus species in a single step by using a single cell line.

Furthermore, viruses from different species are unlikely to initiate recombination events with each other and hence are unlikely to take any part in diversification. However, the culturing together of viruses from more than one serotype from any given viral species may lead to inter-serotype recombination and this may create sufficient new regions of homology to allow recombination between species.

In order to maximise library diversity, therefore, the process of the invention provides for viruses of at least two serotypes from a first species to be cultured together on an appropriate cell line or lines in order to promote intra-species recombination; and only then culturing the (recombined) viruses from the first species with one more viruses from a second (different) species, in order to promote different intra-species recombination events, and inter-species recombination. It is particularly advantageous for at least two virus serotypes from the second species to be or have previously been cultured together in order to promote intra-species recombination within the second species. During or following this process, the resultant recombined viruses may be subjected to further diversification (e.g. mutagenesis) processes.

The present invention therefore describes new approaches to maximise viral library diversity.

Diversity in the viral genome will enable the development of new viruses with new properties, which could be used for applications such as highly potent, selective oncolytic viruses, vaccines, anti-viral therapies and gene therapy vectors.

In a first aspect, there is provided a process for producing a library of viruses, the process comprising:
(a) a first culturing step, comprising culturing together, on one or more cell lines, viruses of at least two different serotypes from a first species of double-stranded DNA virus, and
(b) combining
   (i) viruses obtained from Step (a), with
   (ii) viruses of at least two different serotypes of the same species
   from each of one or more further species of double-stranded DNA viruses, wherein the first species of double-stranded DNA virus and each further species of double-stranded DNA virus are all different species in the same family or same genus of double-stranded DNA viruses;
   to produce a library of viruses;
      and optionally
(c) a second culturing step, wherein the viruses which are combined in Step (b) are cultured together on one or more cell lines; and
(d) combining viruses or portions thereof obtained after Step (c), and/or isolating a plurality of viruses therefrom,
   to produce a library of viruses.

In some embodiments, in Step (b)(ii), for each species of the one or more further species of double-stranded DNA virus, the viruses of different serotypes from that species are ones that have previously been cultured together, wherein viruses of different species were previously cultured independently.

In some preferred embodiments, the process of the invention comprises the steps:
(a) a first culturing step, comprising
   (i) culturing together, on one or more cell lines, viruses of at least two different serotypes from a first species of double-stranded DNA virus; and
   (ii) culturing, on one or more cell lines, viruses of at least two different serotypes of the same species from each of one or more further species of double-stranded DNA viruses,
      wherein, for each species of double-stranded DNA virus, viruses of different serotypes of the same species are cultured together, and viruses of different species are cultured independently; and
(b) combining
   (i) viruses from Step (a)(i), and
   (ii) viruses from Step (a)(ii).

In some embodiments, Step (b) additionally comprises combining viruses from (i) and (ii) with viruses from:
(iii) the first species of double-stranded DNA virus;

(iv) one or more wild-type viruses of the same family, genus or species as the first species of double-stranded DNA virus;
(v) one or more of the further species of double-stranded DNA viruses; and/or
(vi) one or more wild-type viruses of the same family, genus or species as one of the further species of double-stranded DNA virus.

In some embodiments, Step (d) additionally comprises combining viruses or portions thereof obtained after Step (c) with viruses from:
(i) the first species of double-stranded DNA virus;
(ii) one or more of the further species of double-stranded DNA viruses;
(iii) one or more viruses obtained after culturing Step (a);
(iv) one or more wild-type viruses from the same family, genus or species as the first species of double-stranded DNA virus; and/or
(v) one or more wild-type viruses from the same family, genus or species as one of the further species of double-stranded DNA viruses.

In some embodiments, the viruses are subjected to mutagenesis before, during or after one or more of Steps (a), (b) and/or (c).

Preferably, the double-stranded DNA virus is an adenovirus (i.e. from the family Adenoviridae).

The invention relates to a process for producing a library of viruses, e.g. a mixture of wild-type and chimeric viruses, the process comprising Steps (a) and (b) and optionally Steps (c) and (d), as defined herein.

The process of the invention uses double-stranded DNA viruses. Preferably, the double-stranded DNA virus is one selected from the group consisting of Adenoviridae, Asfarviridae, Polyomaviridae, Herpesviridae, Poxviridae and Papillomaviridae families. More preferably, the double-stranded DNA virus is selected from Adenoviridae, Herpesviridae and Poxviridae families. Most preferably, the viruses are from the family Adenoviridae.

The first and further double-stranded DNA viruses are all from the same family or genus. In some embodiments, the first and further double-stranded DNA viruses are all from the same genera within the same family. In other embodiments, the first and further double-stranded DNA viruses are from one or more different genera within the same family.

As used herein, “adenovirus” (also abbreviated herein to “Ad”) refers to those viruses belonging to the family Adenoviridae, included in any one of the currently known five genera: Mastadenovirus, Aviadenovirus, Atadenovirus, Siadenovirus and Ichtadenovirus. Preferably, the adenoviruses are from the genus Mastadenovirus; this includes all human serotypes. In one embodiment, the adenoviruses are human adenoviruses.

At present more than 60 antigenic types or “serotypes” of human adenoviruses have been described and these serotypes have been classified into seven species, i.e. Ad species A-G, on the basis of their physical, chemical and biological properties (as described, for example, in Wold et al. Current gene therapy vol. 13, 6 (2013): 421-33).

Thus adenoviral species as used herein refers to those currently-known Ad groups A-G, as well as any identified in the future.

In one embodiment, therefore, the adenovirus species are species of human adenoviruses selected from the group consisting of AdA, AdB, AdC, AdD, AdE, AdF and AdG. In some embodiments, the species of human adenoviruses are selected from the group consisting of AdB, AdC, AdD, AdE, AdF and AdG.

The serotypes which fall within each of these adenovirus species are given below: AdA comprises Ad12, Ad18 and Ad31. AdB comprises Ad3, Ad7, Ad11, Ad14, Ad16, Ad21, Ad34, Ad35, Ad50 and Ad55. AdC comprises Ad1, Ad2, Ad5, Ad6 and Ad57.

AdD comprises Ad8, Ad9, Ad10, Ad13, Ad15, Ad 17, Ad19, Ad20, Ad22, Ad23, Ad24, Ad25, Ad26, Ad27, Ad28, Ad29, Ad30, Ad32, Ad33, Ad36, Ad37, Ad38, Ad39, Ad42, Ad43, Ad44, Ad45, Ad46, Ad47, Ad48, Ad49, Ad51, Ad53, Ad54 and Ad56. AdE comprises Ad4. AdF comprises Ad40 and Ad41. AdG comprises Ad52. The references herein to different Ad serotypes include all different strains or variants of those serotypes.

In other embodiments, the double-stranded DNA virus is from the family Herpesviridae or Poxviridae. Preferably, the Herpesviridae is of the subfamily Alphaherpesvirinae, Betaherpesvirinae or Gammaherpesvirinae. Preferably, the Herpesviridae is from the subfamily alphaherpesvirinae. Preferably, the alphaherpesvirinae is of the genus Iltovirus, Mardivirus, Simplexvirus, Scuta virus or Varicellovirus.

Preferably, the herpesviruses are from the genus Simplexvirus. Herpes simplex viruses exist as two major species: Herpes Simplex Virus type 1 (HSV-1) and Herpes Simplex Virus type 2 (HSV-2). The different types of herpesvirus are generally referred to in the art as “strains” rather than “serotypes”. However, it will be understood that, in the context of the current invention as it applies to herpesvirus, the terms “serotype” and “serotypes” will encompass “strain” and “strains”, respectively.

For the purposes of this invention, HSV strains are classed as those that differ by greater than 0.5% of the viral genome (see Davison A J. Overview of classification. In: Arvin A, Campadelli-Fiume G, Mocarski E, et al., editors. Human Herpesviruses: Biology, Therapy, and Immunoprophylaxis. Cambridge: Cambridge University Press; 2007. Chapter 1).

Strains of herpes virus include but are not limited to HF10, F E06, H129, 17, KOS, KOS63, KOS79 and JS-1.

Preferably, the Poxviridae is selected from one of the following subfamilies or genera: Chordopoxvirinae, Avipoxvirus, Capripoxvirus, Centapoxvirus, Cervidpoxvirus, Crocodylidpoxvirus, Leporipoxvirus, Macropopoxvirus, Molluscipoxvirus, Mustelpoxvirus, Orthopoxvirus, Oryzopoxvirus, Parapoxvirus, Pteropopoxvirus, Salmonpoxvirus, Sciuripoxvirus, Suipoxvirus, Vespertilionpoxvirus, Yatapoxvirus, Entomopoxvirinae, Alphaentomopoxvirus, Betaentomopoxvirus and Gammaentomopoxvirus.

More preferably, the Poxviridae are from the genus Orthopoxvirus. Preferably, the Orthopoxvirus is from the species Abatino macacapox virus, Akhmeta virus, Camelpox virus, Cowpox virus, Ectromelia virus, Monkeypox virus, Raccoonpox virus, Skunkpox virus, Taterapox virus, Vaccinia virus, Variola virus or Volepox virus, more preferably a vaccinia virus.

The different types of vaccinia virus are generally referred to in the art as “strains” rather than “serotypes”. However, it will be understood that, in the context of the current invention as it applies to vaccinia virus, the terms “serotype” and “serotypes” will encompass “strain” and “strains”, respectively. For the purposes of this invention, vaccinia strains are classed as those that differ by greater than 0.5% of the viral genome.

Strains of vaccinia virus include, but are not limited to, Lister strain, Modified vaccinia Ankara, Western Reserve strain, Dryvax (“Wyeth”), Copenhagen, LC16m8, CV-1 and Tian Tan (see Sanchez-Sampedro L, Perdiguero B, Mejias-Pérez E, Garcia-Arriaza J, Di Pilato M, Esteban M. The evolution of poxvirus vaccines. Viruses. 2015; 7(4):1726-1803. Published 2015 Apr. 7. doi:10.3390/v7041726).

The first culturing step comprises culturing together viruses of at least two different serotypes from a first species of double-stranded DNA virus. In this first culturing step, the serotypes which are cultured together are all from the same virus species. The aim of this step is to promote inter-serotype recombination between viruses of the same species.

In the first culturing step, viruses from different species are cultured independently, e.g. in different/separate culture vessels.

The number of serotypes from each species which are cultured together may be 2, 3, 4, 5, 6, 7, 8, 9, 10 or more, preferably at least 3, at least 4 or at least 5 serotypes.

In one embodiment, the at least two serotypes are at approximately equal proportions (i.e. at an equal number of infectious particles or genome copies) within a sample. This may be such that equal proportions of serotypes are represented within the viral subgroup sample used as the starting material for growing on a cell line.

The viruses from each different species are independently cultured on one or more suitable cell lines which allow for growth and replication (preferably optimal growth and/or replication) of viruses of that species. In some embodiments, each virus species is independently (i.e. separately) grown on a single cell line. In other embodiments, each virus species is independently (i.e. separately) grown on a plurality of cell lines. In some embodiments, the plurality of cell lines are grown together (e.g. in the same culture vessel). In other embodiments, the plurality of cell lines are grown independently (e.g. in different culture vessels).

At the end of the first culturing step, viruses which have been cultured on the cell line or plurality of cell lines may be combined (pooled). In each case, viruses from different species are cultured separately.

Cell lines suitable for growing viruses include immortalised cell lines, such as those isolated from naturally-occurring cancers. In one embodiment, the preferred cell line is a cancer cell line. Suitable cancer cell lines include A549, HT29, HEK293, HCT116, MM1S, SKOV3, MMR, JJN3, RPMI-8226 and U266. Suitable cancer cell lines for growing adenovirus serotypes include, for example, A549, HT29, HEK293, HCT116, SKOV3 and MM1S cells. Other suitable cell lines for growing particular viral subgroups will be known by those skilled in the art. In some embodiments, a cell line is grown to sub-confluence prior to viral infection.

In one embodiment, the cell line is a DNA repair deficient cell line, suitably a cell line which is deficient in DNA repair enzymes or DNA damage sensing, for example HCT116 cells.

For each virus serotype, there will be preferred cell lines which promote maximal viral growth and/or recombination for that particular serotype. The preferred cell lines are those expressing the cellular entry receptor for that particular serotype and/or support high levels of viral genome replication (as defined by >200 fold increase in viral genomes as compared to the input at day 0) within 7 days. Those viruses entering and replicating within cells with similar kinetics have greater opportunity to recombine. Methods for measuring maximal viral growth are known in the art; some are described herein.

In another embodiment, the species is AdB and the preferred cell line is A549 or HCT116, most preferably A549. In one embodiment, the species is AdC and the preferred cell line is MM1S, HEK293 or A549, most preferably A549. In another embodiment, the species is AdD and the preferred cell line is HT29 or A549, most preferably HT29.

In the first culturing step, viruses from each different species are independently cultured on one or more suitable cell lines which allow for replication of those viruses. Methods for infecting cell lines with viruses and the growth of viruses on cell lines are well known in the art (e.g. Shashkova E V, May S M, Barry M A. Characterization of human adenovirus serotypes 5, 6, 11, and 35 as anticancer agents. Virology. 2009; 394(2):311-320. doi:10.1016/j.virol.2009.08.038; and Freedman J D, Hagel J, Scott E M, et al. Oncolytic adenovirus expressing bispecific antibody targets T-cell cytotoxicity in cancer biopsies. EMBO Mol Med. 2017; 9(8):1067-1087. doi:10.15252/emmm.201707567).

By viral “growth” or “growing” is meant amplifying the number of virus particles. “Growing” a virus includes infecting a suitable cell line and culturing those infected cells under conditions permitting viral replication. Viral growth may be quantified using techniques such as qPCR to measure the number of viral genomes. Suitably “growing” includes “passaging” viruses. By “pooling” is meant mixing samples obtained from separate reactions. In this instance, “pooling” refers to the mixing of resultant amplified viral samples.

By “grown independently” is meant that each viral group is grown on a cell line alone. For example, AdC viruses are grown on a cell line alone separately from AdD viruses.

Prior to infection of the cell line with the viruses, the cell line may be grown to sub-confluence. In some embodiments, the cells are grown to approximately 70% confluency. For example, cells may be grown at a density of about 1×10⁶ cells in T25 culture flasks.

Infection is preferably at a virus particle-per cell ratio of 50-1000 vp/cell, more preferably up to 500 vp/cell. Advantageously, this particle per cell ratio is optimised to promote recombination between serotypes. These ratios are described herein, in particular for AdB, AdC, AdD, AdE, AdF or AdG viral libraries.

Alternatively, cells are infected at an MOI (multiplicity of infection) of 1-100, preferably at an MOI greater than 10.

Preferably, the viruses are cultured on appropriate cell lines in the first culturing step under conditions which promote inter-serotype recombination. Preferably, those conditions comprise multiple rounds of viral replication, wherein multiple viruses are generated in each round of viral replication.

In some preferred embodiments, up to 30 rounds of viral replication are allowed to occur in the first or second culturing during Step (a) or Step (c), preferably at least 1, 2, 3, 4, 5 or at least 6 rounds of viral replication, in each passage. For example, 5 rounds of replication are allowed over a 5-7 day period.

In one embodiment, the viruses grown on cell lines (preferably cancer cell lines) are harvested after 1, 2, 3, 4 5, 6, 7, 8, 9 or 10, days, preferably after 3 to 7 days.

In some embodiments, the viruses are passaged 1, 2, 3, 4, 5, 6, 7, 8, 9 or 10 times, preferably 4-6 times during the first or second culturing step.

For example, a number of rounds of “passaging” are involved to “grow” a virus to increase the number of viral genomes. Suitably, one round of “passaging” involved infecting cells with a virus at a suitable virus particle:cell ratio; growing the cells under suitable conditions to allow the virus to exert its full cytopathic effect (i.e. in the case of adenovirus, to lyse the host cell and release viral particles); and collecting the cells and/or supernatant comprising the virus particles and using this for another round of “passaging”. In some embodiments, the cells and/or supernatant may be subjected to “freeze thaw”.

“Freeze-thaw” as used herein refers to the process of freezing the harvested cells and/or supernatant from virally-infections and thawing it, prior to it being used as a starting material for the subsequent passage on sub-confluent cultures. “Freeze-thaw” processes result in an optimal viral particle release from virally-infected cells. Suitably, the round of freeze-thaw in accordance with the invention is preferably, at least 1 round, or 2 rounds or 3 rounds.

In some preferred embodiments, the cell line cells are grown to sub-confluence, followed by infection of 50-1000 vp/cell, preferably 100-500 vp/cell or at between 1-100 MOI.

In some preferred embodiments, the viruses are passaged about 5 times, each after 3-7 days. In some other preferred embodiments, the viruses are passaged 2-6 times, each after 2-6 days, in the first and/or second culturing step.

In some preferred embodiments of the invention, Step (a) comprises:
(a) a first culturing step, comprising
   (i) culturing together, on one or more cell lines, viruses of at least two different serotypes from a first species of double-stranded DNA virus; and
   (ii) culturing, on one or more cell lines, viruses of at least two different serotypes of the same species from each of one or more further species of double-stranded DNA viruses, wherein, for each species of double-stranded DNA virus, viruses of different serotypes of the same species are cultured together, and viruses of different species are cultured independently.

In this embodiment, for each species, viruses of at least two different serotypes from the same species are cultured together, thus promoting intra-species recombination. Viruses from a number of different species of double-stranded DNA virus are each cultured independently (i.e. separately) or essentially independently on one or more cell lines (and then optionally pooled). The cell line or lines are preferably selected for each species of virus such that that species of virus grows optimally on that cell line or cell lines.

Each cell line culture comprises viruses of only one species or substantially only of one species; each culture comprises the majority of viruses of one species; or each culture comprises viruses which grow optimally on that cell line. Trace amounts or insignificant amounts of viruses of other species may be present in each cell-line culture.

In this embodiment, viruses of at least two different serotypes of the same species from each of one or more further species of double-stranded DNA viruses are cultured. The term “one or more further species” may include 1, 2, 3, 4, 5, 6, 7, 8, 9, 10 or more species of double-stranded DNA viruses, preferably 1-5, and more preferably 3-5 further species.

Step (a) is performed before Step (b).

Step (b) comprises:
(b) combining
   (i) viruses obtained from Step (a), with
   (ii) viruses of at least two different serotypes of the same species from each of one or more further species of double-stranded DNA viruses,
      wherein the first species of double-stranded DNA virus and each further species of double-stranded DNA virus are all different species in the same family or same genus of double-stranded DNA viruses.

The viruses which are obtained from Step (a) and which are used in Step (b) will in general be all or essentially all of the viruses obtained from Step (a) or samples thereof.

The term “one or more further species” may include 1, 2, 3, 4, 5, 6, 7, 8, 9, 10 or more species of double-stranded DNA viruses, preferably 1-5, and more preferably 3-5 further species.

In some embodiments, in Step (b)(ii), for each species of the one or more further species of double-stranded DNA virus, the viruses of different serotypes from that species are ones that have previously been cultured together, wherein viruses of different species were preferably previously cultured independently.

In this embodiment, viruses of different serotypes of the same species have previously been cultured together (e.g. on one or more cell lines) in order to promote intra-species recombination. Viruses of different species have preferably previously been cultured independently (i.e. separately) or substantially independently. For example, each culture comprised viruses of only one species or substantially only of one species; each culture comprised the majority of viruses of one species; or each culture comprised viruses which grew optimally on the cell line.

In some embodiments, Step (b) additionally comprises combining viruses from (i) and (ii) with viruses from:
(iii) the first species of double-stranded DNA virus;
(iv) one or more wild-type viruses of the same family, genus or species as the first species of double-stranded DNA virus;
(v) one or more of the further species of double-stranded DNA viruses; and/or
(vi) one or more wild-type viruses of the same family, genus or species as one of the further species of double-stranded DNA virus.

In particular, Step (b) additionally comprises combining viruses from (i) and (ii) with other viruses from a species of the same genus as the first species of double-stranded DNA viruses (including viruses of a single serotype of that species of the same genus).

Preferably, at least 1% (e.g. 1-5% or 5-10%) of these additional viruses are included.

Step (c), which is optional, comprises:
(c) a second culturing step, wherein the viruses which are combined in Step (b) are cultured together on one or more cell lines.

Whilst the aim of the first culturing step was to promote intra-species recombination, the aim of the second culturing step is to promote inter-species recombination. The cell lines which are used in the second culturing step may be one or more of the cell lines described above in the context of the first culturing step. The parameters for infection and cell culturing (e.g. MOI, rounds of viral replication, duration of culturing and passaging) in the second culturing step are the same as those give above for the first culturing step, mutates mutandis.

In particular, viruses from the first species and each further species are cultured together on one or more suitable cell lines which allow for growth and replication (preferably optimal growth and/or replication) of viruses of those species. In some embodiments, these viruses are grown together on a single cell line. In other embodiments, these viruses are grown together on a plurality of cell lines. In some embodiments, the plurality of cell lines are grown together (e.g. in the same culture vessel). In other embodiments, the plurality of cell lines are grown independently (e.g. in different culture vessels).

The process of the invention may optionally additionally comprise the step:
(d) combining viruses or portions thereof obtained after Step (c), and/or isolating a plurality of viruses therefrom,
   to produce a library of viruses.

In some embodiments, Step (d) additionally comprises combining viruses or portions thereof obtained after Step (c) with viruses from:
(i) the first species of double-stranded DNA virus;
(ii) one or more of the further species of double-stranded DNA viruses;
(iii) one or more viruses obtained after culturing Step (a);
(iv) one or more wild-type viruses from the same family, genus or species as the first species of double-stranded DNA virus; and/or
(v) one or more wild-type viruses from the same family, genus or species as one of the further species of double-stranded DNA viruses.

Methods of isolating viruses are well known in the art (as described in Cromeans T L, Lu X, Erdman D D, Humphrey C D, Hill V R. Development of plaque assays for adenoviruses 40 and 41. J. Virol. Methods. 2008; 151(1):140-145. doi:10.1016/j.jviromet.2008.03.007).

Preferably, at least 1c/o (e.g. 1-5% or 5-10%) of these additional viruses are included.

In some embodiments, the viruses are subjected to mutagenesis before, during or after one or more of the steps of the invention. This further promotes diversification within the virus library.

Such further diversification processes are well known in the art (e.g. Wechman S. L., et al. “Development of an Oncolytic Adenovirus with Enhanced Spread Ability through Repeated UV Irradiation and Cancer Selection”. Viruses. 2016; 8:6. doi: 10.3390/v8060167).

Preferably, the mutagenesis is by UV irradiation. This may be used to induce mutations within the viral genomes and/or to induce DNA breaks which may encourage recombination between the viruses.

As used herein, the term “library of viruses” refers to a collection or mixture of viruses having different genome sequences. In some embodiments, a “library” may comprise viruses having a wild type viral genome sequence, including different wild type subgroups or serotype genome sequences. For example, a library may include viral genomes which are made up of a combination or combinations of nucleic acid sequences from different viral genomes.

The viral genome sequences may be nucleic acid sequences.

A “library” may also comprise wild type genome sequences in combination with modified, recombinant or mutated viral genome sequences, wherein the viral genome sequence differs from the naturally-occurring or “wild-type” genome sequence.

Preferably, the process of the invention generates a library having a diversity of at least 1 new recombinant viral genome (comprising at least one new recombination event), preferably more than 1 new recombinant viral genome, when compared to the viruses used as a starting point in the process as described herein. Bioinformatics analysis may be used to measure new recombination events and to align the sequenced viral library output against published (GenBank) wild-type human viral serotypes. The higher the number of mutations/DNA swaps or recombination events, the greater the diversity in the library.

As used herein, “new recombination event” means the recombination of nucleic acids from one viral serotype to another viral serotype wherein the resulting viral genome differs from the naturally-occurring or “wild-type” genome sequence by at least one nucleotide.

In another aspect, the invention provides a library which is obtained by or obtainable by a process in accordance with any aspect or embodiment of the invention.

The invention also provides a chimeric virus, preferably a chimeric adenovirus, obtained by or obtainable by a process in accordance with any aspect or embodiment of the invention.

Various further aspects and embodiments of the present invention will be apparent to those skilled in the art in view of the present disclosure. All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments, it is not limited to the disclosed embodiments and that alternative embodiments could be constructed without departing from the scope of the invention as defined in the appended claims.

The term “comprising” encompasses “including” as well as “consisting” e.g. a composition “comprising” X may consist exclusively of X or may include something additional e.g. X+Y.

All numbers and ranges disclosed above may vary by some amount. Whenever a numerical range with a lower limit and an upper limit is disclosed, any number and any included range falling within the range is specifically disclosed. In particular, every range of values (of the form, “from about A to about B,” or, equivalently, “from approximately A to B,” or, equivalently, “from approximately A-B”) disclosed herein is to be understood to set forth every number and range encompassed within the broader range of values.

The disclosure of each reference set forth herein is specifically incorporated herein by reference in its entirety.

### BRIEF DESCRIPTION OF THE FIGURES

FIG. 1.1. Virus genome replication measured by QPCR (n=4) at 0, 1, 3, 6 days post-infection of A549, HT29, HEK293, HCT116, SKOV3 and MM1S cells with 200 vp/cell of AdB, AdC or AdD virus libraries.

FIG. 1.2. Shifting proportions of Ad species across multiple passages demonstrates a collapse of virus diversity when Ad species are passaged together in A549 cells.

FIG. 1.3. Pooling cell lines may aid diversification.

FIG. 2.1. Virus genomes from three rounds of infection were quantified by QPCR. HT29 cells were infected with 200 vp/cell of AdC and AdD virus libraries, or co-infected with 200 vp/cell AdC or AdD virus libraries for 3 rounds of infection.

FIG. 2.2. Viral competition in different cell lines results in unique species distributions.

FIG. 2.3. Prior art methods to create virus libraries results in a loss of diversity.

FIG. 3.1. A higher rate of inter-species chimeras are detected following the Step-wise Diversification Process (Stage 1) than Single Stage diversification (prior art). Stage 1 of the Step-wise Diversification process is the sum of all Ad-B, C, D chimeric viruses from the output of each cell line combined. % chimeric reads is the percentage of all next generation sequencing (NGS, Illumina) reads that evince a recombination breakpoint.

FIG. 3.2. The Step-wise Diversification Process promotes broad participation of adenovirus chimera parent pairs compared to Single Stage Diversification (prior art).

FIG. 3.3. The Step-wise Diversification Process creates chimeras with recombination sites spanning the genome. The positions of AdC chimera recombination sites span the genome for the Step-Wise Diversification Process, whereas none were detected for Single Stage Diversification (Prior art).

FIG. 3.4. Quantity of sequencing reads demonstrating AdB chimera recombination sites across the adenovirus genome. Analysis of two representative Ad-B serotypes (denoted as AdB.1 and AdB.2 in figure below) and their chimeras highlights the levels of diversity created using each approach. Evidence of different rates and types of AdB.1/AdB.2 recombination events occurring across the virus genome during Stage 1 and 2 of the diversification process is demonstrated. Ad genomic deletions were detected to a greater extent during Stage 1 than Stage 2. (i) Data was generated using synthetic long read sequencing approaches (reconstruction of IIlumina reads from the same virus genome informed by barcode tagging). (ii) Data was generated using short Illumina reads.

FIG. 4.1. Plot of AdD chimera rates detected following passaging the AdD libraries in HT29 cells or HCT116 cells. Chimeras were detected using synthetic long read sequencing.

FIG. 4.2. Plot of AdB and AdC chimera rates detected following passaging the AdB/C libraries in A549 cells or HCT116 cells. Chimeras were detected using short read sequencing.

FIG. 4.3. Quantity of sequencing reads demonstrating AdB chimera breakpoints across the adenovirus genome following infection of Ad-B libraries in A549 and HCT116 cells.

FIG. 5.1. Sequence similarity between Ad serotypes across the genome.

FIG. 5.2. Sequence similarities between HSV1 isolates. Horizontal dashed line represents 0.5% sequence divergence (0.25% along each branch from their common ancestor) and boxes indicate isolates assigned to the same virus strain.

FIG. 5.3. Sequence similarities across the genome compared to HSV-1 strain 17.

FIG. 5.4. Sequence similarities between Vaccinia strains and other Orthopoxvirus species. Horizontal dashed line represents 0.5% sequence divergence (0.25% along each branch from their common ancestor) and boxes indicate clones assigned to the same virus strain. (WR=Western Reserve, Cop=Copenhagen).

FIG. 5.5. Sequence similarities across the genome compared to Vaccinia Western Reserve.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Preferential Growth of Viruses in Different Cell Lines

Virus Preparations

Wild-type human adenovirus (Ad) serotypes from Ad species B (AdB), AdC, AdD, AdE, AdF and AdG were included in this study (Robinson C M et al., Molecular evolution of human adenoviruses. Sci. Rep. 2013; 3:1812). Each Ad serotype was plaque-purified and single isolates were verified by whole genome sequencing, or Sanger sequencing over a 1 kb E2B region and found to align correctly to the corresponding Genbank ID entry. Viruses were amplified and titred by TCID50 on HEK293 cells. Equal infectious particles of each serotype were pooled according to their Ad species (e.g. Ad1, Ad2, Ad5, Ad6=AdC virus library) and purified by double-banding using CsCl gradients to generate species specific Ad libraries (e.g. AdB, AdC, AdD libraries).

Understanding Viral Replication Kinetics

Time-course infections were performed with AdB, AdC and AdD virus libraries in a panel of human cancer cell lines (A549, HT29, HEK293, HCT116, SKOV3 and MM1S cells, obtained from the ATCC). A549, HT29, SKOV3 and HEK293 cells were cultured in DMEM with 10% FBS at 37° C., 5% CO₂. HCT116 and MM1S cells were cultured in RPMI-1640 with 10% FBS at 37° C., 5% CO₂. Cells were seeded 24 hrs prior to infection with AdB, AdC or AdD virus libraries and incubated at 37° C., 5% CO₂. Samples (virally-infected cells and supernatant combined) were harvested for virus genome replication studies at 0, 1, 3, 6-7 days post infection. Virus genomes were quantified by qPCR using Ad species-specific primers (Life Technologies):

| | AdB Forward |
|---|---|
| (SEQ ID NO: 1) | |
| | GAGTTGGCTTTAAGTTTAATGAGC, |
| | AdB Reverse |
| (SEQ ID NO: 2) | |
| | TGAGGCCTGATAAACAGTAT, |
| | AdC Forward |
| (SEQ ID NO: 3) | |
| | GCTTAATGACCAGACACCGT, |
| | AdC Reverse |
| (SEQ ID NO: 4) | |
| | GGTATATGCAAAGGTGGCA, |
| | AdD Forward |
| (SEQ ID NO: 5) | |
| | GGGATGATGACCGAGCTG, |
| | AdD Reverse |
| (SEQ ID NO: 6) | |
| | CAGACATGCCTGCTACAT; |

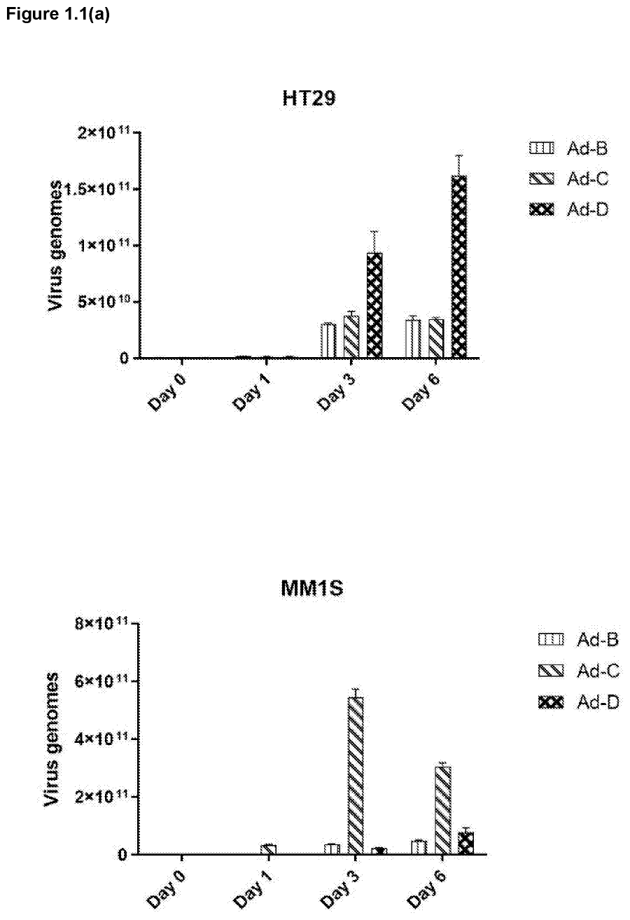

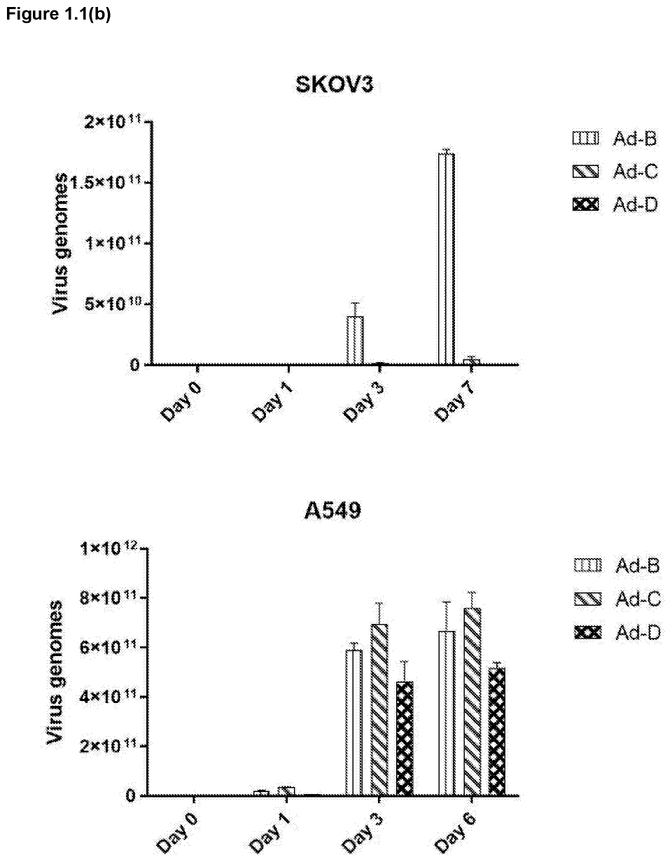

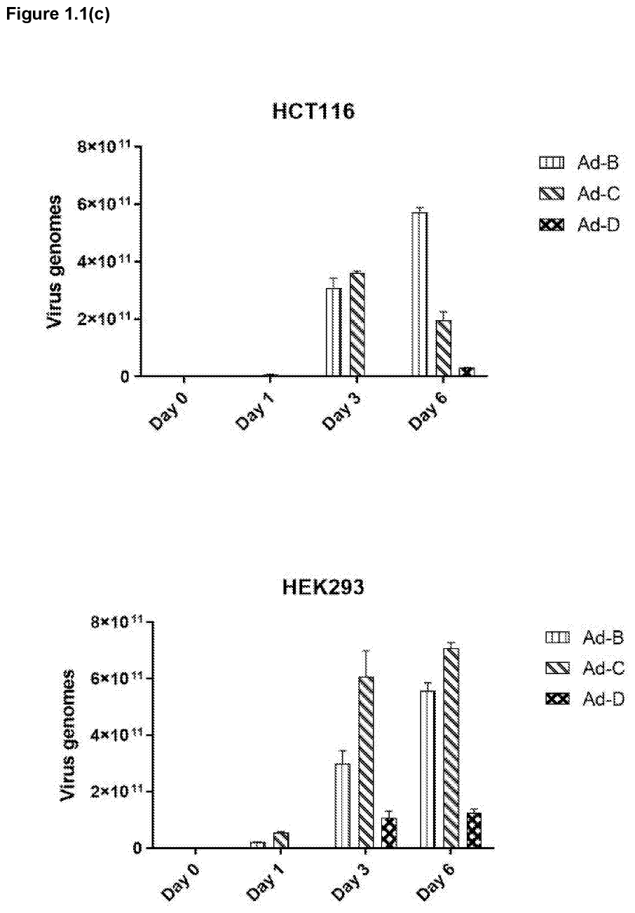

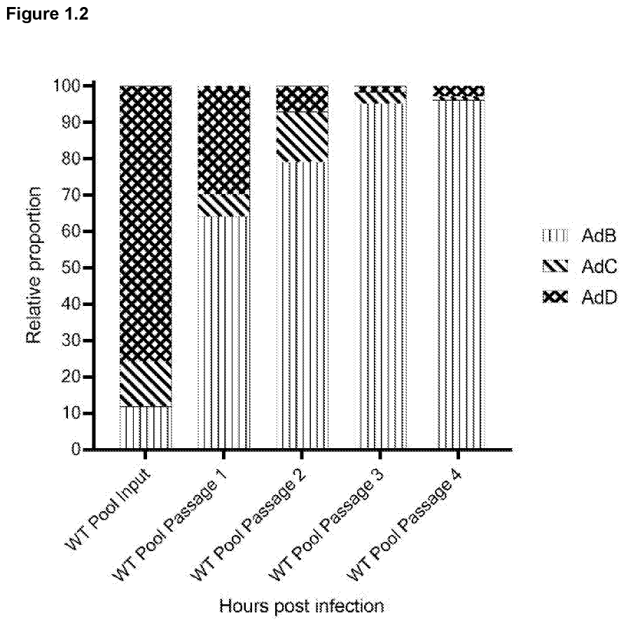

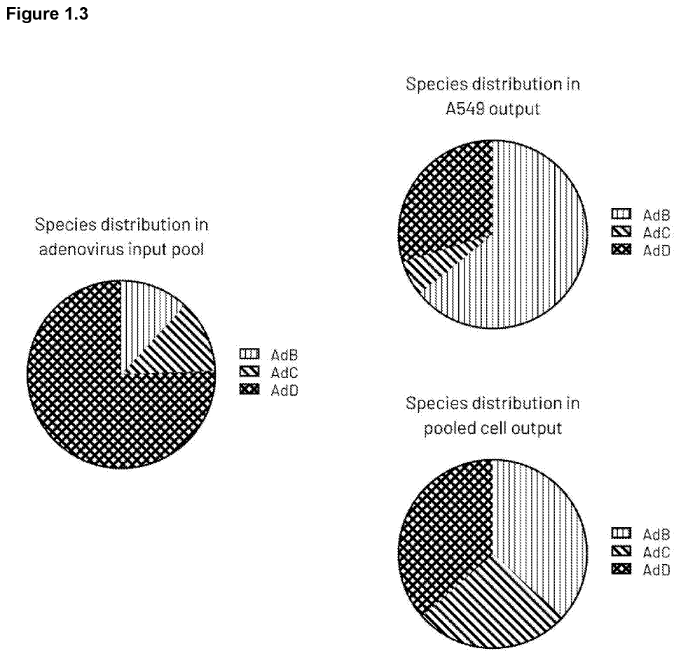

and data represented as total virus genomes per Ad species over time (FIG. 1).

The data in FIG. 1.1 demonstrates that adenoviruses from different species (for example AdB, C, D) tend to show preferable infection and/or replication efficiencies in different cell lines. For example, AdC viruses replicated much more rapidly in MM1S and HEK293 cells than AdD viruses. AdC viruses reached maximal genomes at 3 days, while AdD viruses remained >10 fold lower, indicating dramatically reduced opportunities for AdD recombination events in these cell lines, in this time frame, compared to AdC viruses.

Of the cell lines tested, HEK293 cells preferentially support AdC>AdB>AdD replication; MM1S support AdC>AdD>AdB; A549 support AdC/B>AdD; HCT116 support AdB>AdC>AdD; SKOV3 support AdB>AdC/D; HT29 support AdD>AdB/C replication at 6 days. Overall A549 had the highest levels for viral replication and HT29/SKOV3 cells supported the lowest levels.

An input virus library consisting of a pool of wild-type (WT) adenoviruses from three species was assessed for species distribution across multiple passages. An equal titre of each WT adenovirus was added to the input library (more Ad-D viruses than Ad-B/C exist in nature) hence the species distribution in FIG. 1.2. This library was infected at a high MOI in A549 cells and passaged up to 4 times, each time on a fresh cell monolayer at a high MOI. At each stage, outputs were analysed via qPCR for titres of AdB, AdC & AdD species, with the relative proportion of each species versus the total titre of the three species plotted for each passage. Despite spiking in the input WT virus library at each passage, the distribution of adenovirus species shifted dramatically towards AdB by the second passage.

To address the dominance of a single Ad species and collapse of viral diversity observed in FIG. 1.3. multiple different cell lines were seeded into the same culture vessel and infected with the AdB, AdC and AdD WT pool. Following a single passage, the species distribution of viruses released into the supernatant was analysed via qPCR and plotted for each experimental condition. Unlike in A549 cells, the pooled cell output had relatively equal distribution across the number of component species indicating the importance of using the outputs of different cell lines to create virus diversity.

### Example 2: Viral Competition in HT29 Cell Lines

HT29 cells were seeded at 70% confluence in T25 flasks in 10% media and incubated at 37° C., 5% CO₂. The next day cells were infected with 200 vp/cell of AdC or AdD virus libraries, or co-infected with 200 vp/cell AdC and AdD virus libraries. Infected cells and supernatants were harvested at signs of CPE post infection, exposed to 1 freeze-thaw cycle and then used as the inoculum for the next round of infection on HT29 cells. This process was repeated three times. Virus genomes in the supernatants from the third round of infection were quantified by qPCR using Ad species specific primers:

| | AdC Forward |
|---|---|
| (SEQ ID NO: 3) | |
| | GCTTAATGACCAGACACCGT, |
| | AdC Reverse |
| (SEQ ID NO: 4) | |
| | GGTATATGCAAAGGTGGCA, |
| | AdD Forward |
| (SEQ ID NO: 5) | |
| | GGGATGATGACCGAGCTG;, |
| | AdD Reverse |
| (SEQ ID NO: 6) | |
| | CAGACATGCCTGCTACAT. |

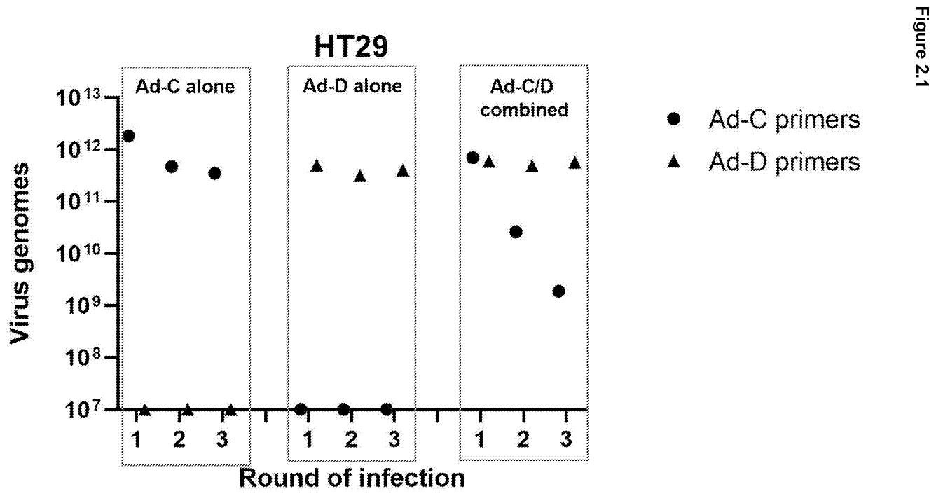

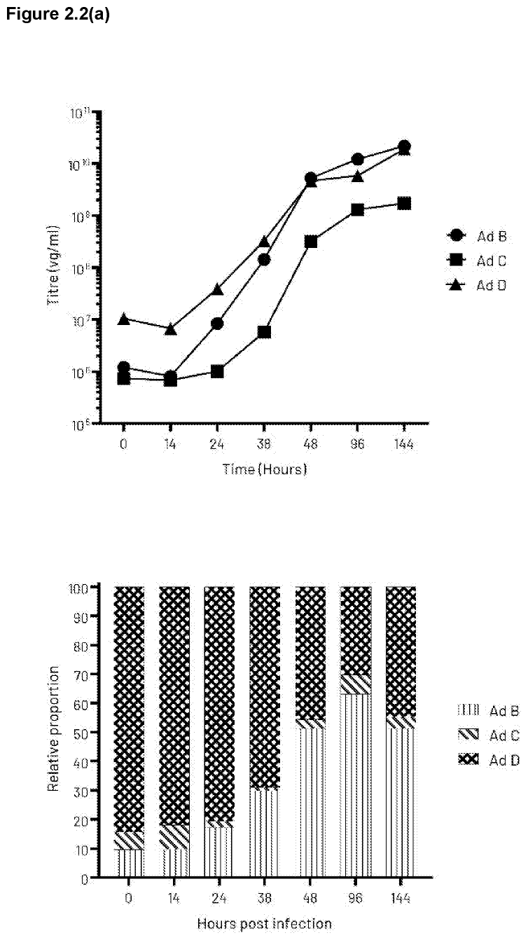

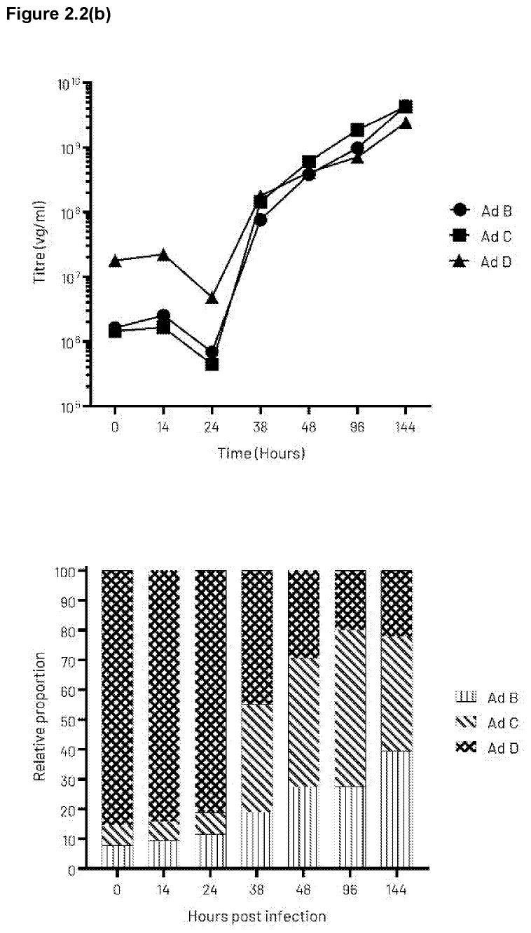

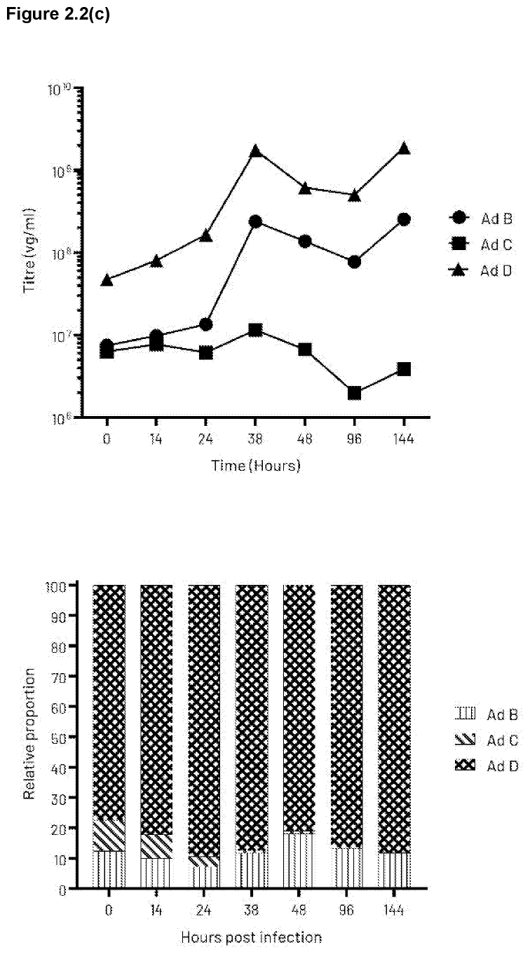

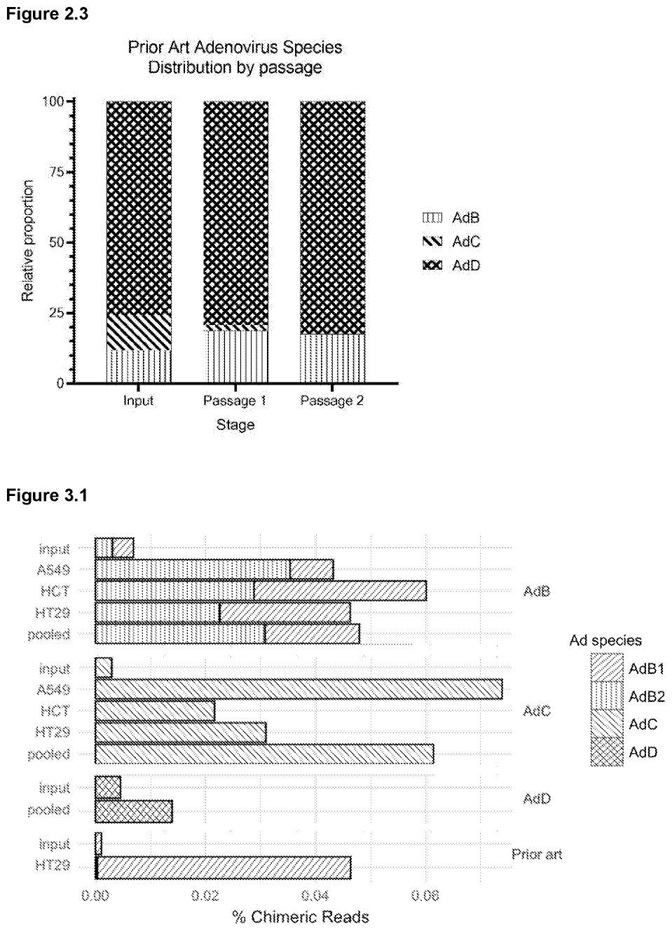

The data is shown in FIG. 2, and demonstrates when cells are co-infected with different Ad species, one virus species will outgrow the other over repeated rounds of infection; and virus genomes in the independently-infected cells will be in significantly higher quantities than when cells are co-infected with AdC and AdD libraries i.e. more AdC viruses were recovered from each round in the absence of other species. Because only those viruses entering cells or replicating at the same time will have a chance of recombining, in order to generate a diverse library, with representatives from as many serotypes and species as possible, the data in FIGS. 1 and 2 demonstrates that each adenovirus species should be grown separately on its preferred cell line (i.e. a cell line which permits maximum viral genome amplification for a given Ad species). The virus species amplified in this way can then be pooled to provide a library containing all wild-types, recombinants and variations thereof.

To explore the transcription and viral release kinetics of the AdB, AdC and AdD species, a time course was set up with three cell lines (A549, HCT and HT29). Cells were seeded and infected at a high MOI with a viral pool formed from the AdB, AdC and AdD WT species weighted by equal serotype termed the WT pool input (similar to FIG. 1.2). Infections were set up at an appropriate vg/cell for the cell line infected. Wells of each cell line were harvested at multiple timepoints post infection (14 hrs, 24 hrs, 38 hrs, 48 hrs, 96 hrs and 144 hrs) as well as a sample of the original infection material to use as a 0 hr control. At each timepoint titres of each species were determined within the harvested supernatant by qPCR which is displayed in FIG. 2.2.

The top row of FIG. 2.2 shows supernatant titers following a pooled infection comprising AdB, AdC and AdD virus libraries, weighted by equal serotype. Top row, A, B and C represent virus titres following infections in A549, HCT116 and HT29 cells respectively. Data supports the use of multiple cell lines to promote recombination events in different Ad species due to differences in infection and replication efficiency between the species across different cell lines. Bottom row displays species distribution, calculated by proportion of each AdB, AdC or AdD total genomes relative to the total genomes across all three species. Data supports use of different cell lines due to stark differences in relative proportions between the cell lines.

As a comparison to the prior art method for generating virus libraries by recombination, our input pool of WT viruses (a combination of AdB, C & D libraries) were passaged according to the methods detailed in the prior art (Kuhn et al., 2008, supra). In brief, HT29 cells were infected with a pool of viruses at 200 vg/cell. Output viruses were titred via qPCR and a second round of infection established with the same conditions. Mapping of the Ad species distribution at input, passage 1 and passage 2 reveals an almost total collapse of AdC abundance, removing this group from the pool of available recombination targets. Therefore the use of multiple cell lines in which AdC is able to compete is required to provide the most targets for recombination and therefore maximum diversity.

### Example 3: Comparison of Single-Stage Viral Diversification and Stepwise Viral Diversification Techniques

Single Stage Viral Diversification

3 serotypes from AdB and 1 each from AdC, AdD, AdE and AdF (i.e. using a similar method to Kuhn et al., 2008, supra, to act as comparator)) were pooled and passaged on sub-confluent cultures of HT29 cells in T175 flasks. Cells were infected with 200 vp/cell of the pooled Ads in 2% culture media at 37° C., 5% CO₂. Viral lysates were harvested from these infected cultures at 48-96 hours post-infection, then frozen at −80° C. Virally-infected cells underwent 3 freeze-thaw cycles and the released viruses were used as the infectious inoculum for a subsequent passage on sub-confluent cultures. The viral lysates were harvested at 48-72 hours post-infection from these cultures and underwent 3 freeze-thaw cycles before purification on CsCl density gradients. The purified viruses were deemed the output ‘diversified library’ from this approach.

Stepwise Viral Diversification

Stage 1—To Promote More Intra-Species Recombination Events.

Viral group libraries of AdB (>6×AdB serotypes), AdC (4×AdC) or AdD (>29×10 AdD serotypes) were passaged independently on sub-confluent cultures of cancer cell lines (A549, HT29, HCT116) in 10% culture media at 37° C., 5% CO₂. Cells were seeded 24 hours prior to infection at 60-70% confluence in T25 culture flasks. Cells were infected with a suitable vp/cell of AdB, AdC, or AdD libraries. Upon cytopathic effect (CPE), the released virus for the particular Ad species were harvested. Following one freeze-thaw cycle, clarified supernatants from the first round of virus infection were added to a sub-confluent layer of cancer cells in T75 flasks in 10% culture media; again each Ad species was passaged independently. The volume of supernatant chosen was that which produced signs of CPE in the following round of infection between 2-5 days.

This cycle of infection on T75 flasks was repeated up to 5 times to introduce recombination events within the Ad species. Output virus genomes for each round of infection were quantified by qPCR using species-specific primers. The output from each cell line was pooled on an Ad species-specific basis and where appropriate purified by CsCl density gradients. Together the purified viruses were deemed to be the output ‘diversified library’ from stage 1.

Stage 2—To Provide Opportunity for Novel Intra-Species and Inter-Species Recombination Events.

Equal virus genomes from Stage 1 (i.e. AdB, AdC, AdD wild-types and variants thereof) were pooled. Viral species libraries were passaged together on sub-confluent cultures of cancer cell lines (A549, HT29, HCT116) in 10% culture media. Cells were split 24 hours prior to infection at 60-70% confluence in T75 culture flasks. Cells were infected with a suitable vp/cell of the pooled Stage 1 virus libraries. Upon CPE, the released virus was harvested. Following one freeze-thaw cycle, clarified supernatant from the first round of viral infection was added to a subconfluent layer of cancer cells in T75 flasks in 10% culture media. The volume of supernatant chosen was that which produced signs of CPE in the following round of infection between 2-5 days. This cycle of infection on T75 flasks was repeated up to 5 times to promote intra and inter-Ad species recombination events. The output from each cell line was pooled and where appropriate purified by CsCl density gradients. The purified virus pool was deemed the output ‘diversified library’ from stage 2.

In the virus pool used in the ‘Single step viral diversification’, there were 3 serotypes from AdB and 1 each from AdC, AdD, AdE and AdF. In the ‘Stepwise library diversification’ there were multiple serotypes from AdB, AdC and AdD.

Diversity of a virus library with respect to virus recombination was determined by high throughput next-generation sequencing (NGS) of the virus genomes in that library. Sequences are aligned against a reference set comprising sequences for each known WT virus. Reads mapping to multiple references were confirmed as chimeric using blast searches.

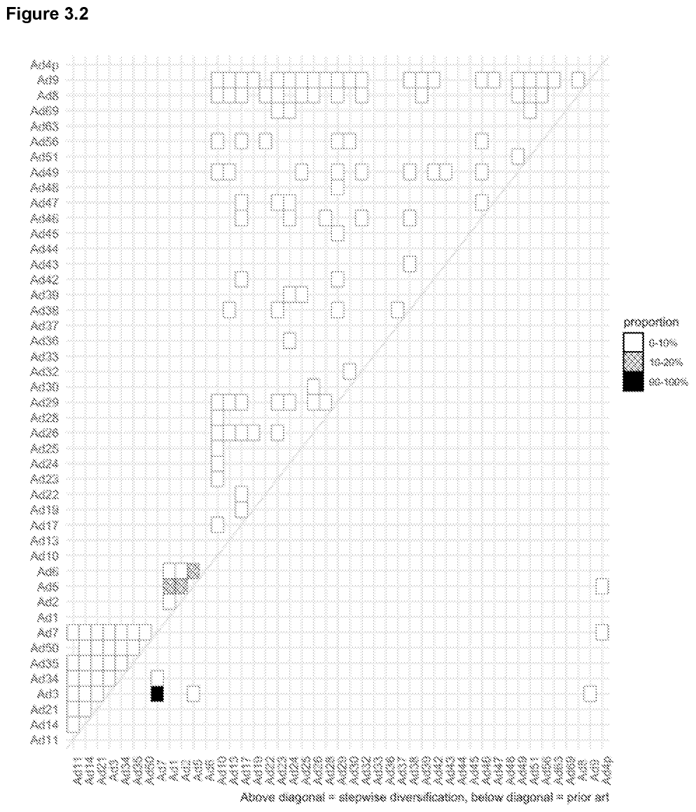

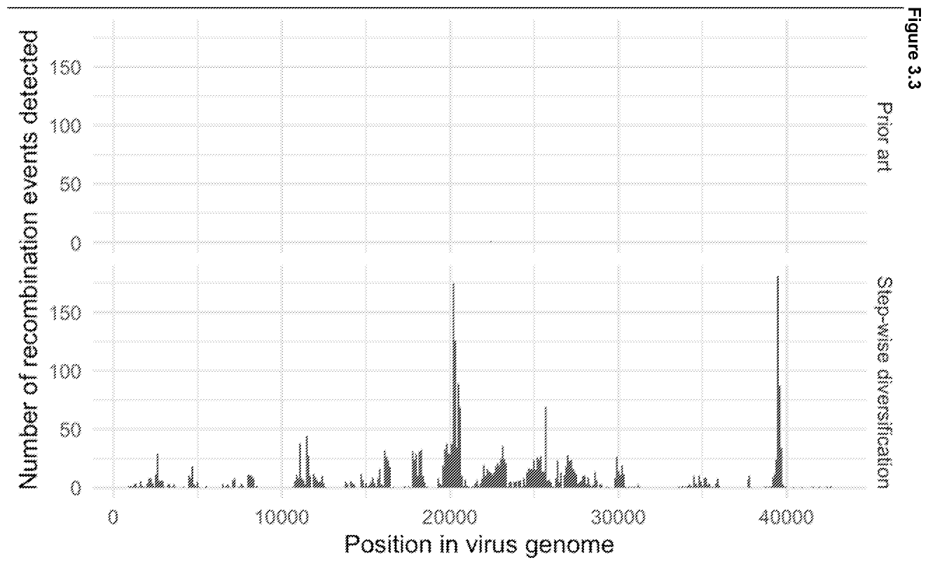

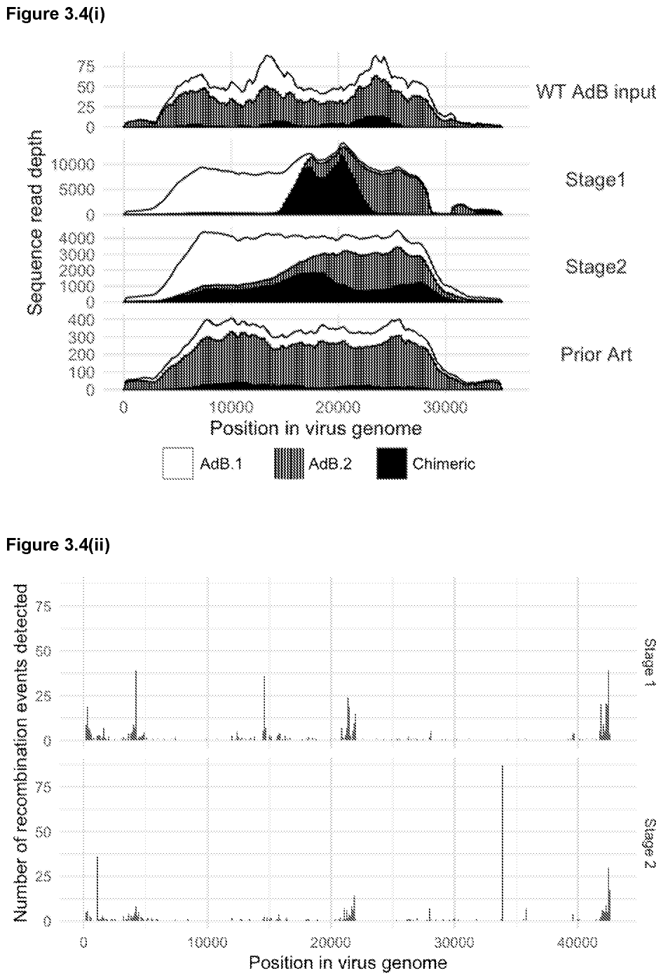

The Step-wise Diversification Process was found to be superior to the prior art method, both in expanding the number and type of virus variants, enabling more variants to participate and preventing dominance of a particular virus group (FIGS. 3.1 and 3.2).

Intra-species Ad chimeras were detected at a higher rate (higher total % chimeric sequence reads, hence higher rate of recombination) following the Diversification Process Stage 1 than were detected using the prior art process or matched Ad-B, Ad-C or Ad-D WT pool inputs (no diversification process applied) (FIG. 3.1). Diversification Process Stage 1 entails passaging of Ad species in different cell lines independently, prior to combining all outputs (i.e. in this case the sum of AdB, C, D chimeras generated in A549, HCT116, HT29). This increases the number of recombinants within each Ad species by co-infections using viruses with larger stretches of sequence homology and similar infection kinetics (i.e. Ads within each species) in their preferred cell type to synchronise infections, prior to combining the outputs with the Stage 2 process and the WT pool input. The approach also enables more Ad serotypes to contribute to recombination events, and thus increases overall library diversity. This is in contrast to the prior art methods in which different Ad species were pooled and used to infect one cell line, resulting in dominance of the Ad-B viruses and collapse of virus diversity (FIGS. 3.1 and 3.2). It should be noted that the percentage of chimeric sequence reads shown, particularly for AdD, is likely to underestimate the total % due to limitations with short read sequencing analysis approaches in homologous viruses.

The Stepwise Diversification Process was found to be superior to the prior art method, enabling virus recombination events distributed across the genome.

The Stepwise Diversification Process (Stage 1) includes at least two different adenovirus serotypes from each species, thereby providing viruses with larger stretches of sequence homology and similar infection kinetics (i.e. Ads within each species) in their preferred cell type to synchronise infections, prior to combining the outputs with the Stage 2 process and the WT pool input. This approach creates recombination sites spanning across the whole virus genome ensuring diverse functional variants (FIG. 3.3). By opening up more of the virus genome in this way, previously unexplored functional virus traits may be revealed which increase the search space to identify the best therapeutic viruses. In contrast, if only using one adenovirus serotype from each species, as is the case of all Ad species aside from Ad-B in the prior art, there are very few chances for recombination events to occur; consequently, no Ad-C chimeras were detected using the prior art approach.

Different types of virus recombination events and adenovirus variants may be produced during Stage 1 and Stage 2 of the Step-wise Diversification Process. Therefore combining the outputs of both Stages 1 and 2 with the input viruses can further enhance virus library diversity.

### Example 4: Some Cell Types have an Increased Propensity for Allowing Viral Recombination Events

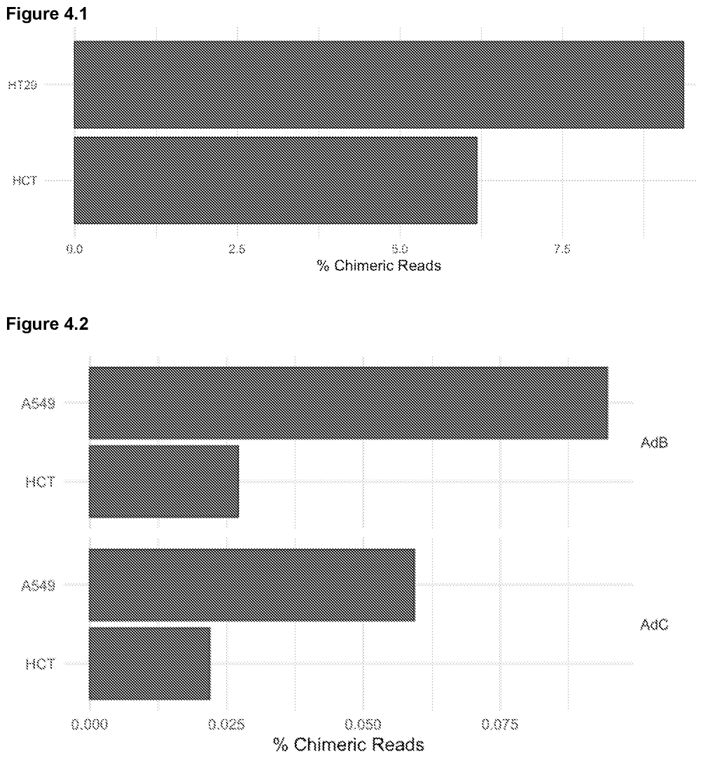

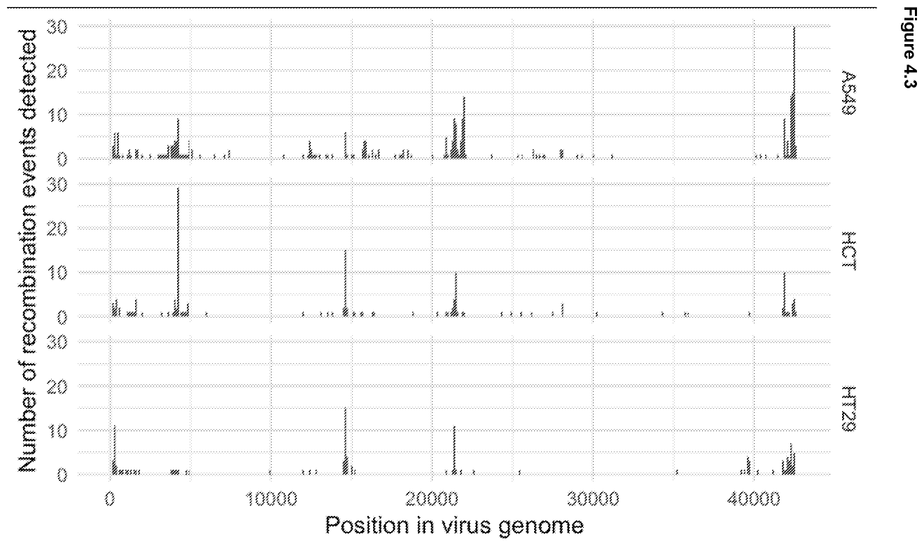

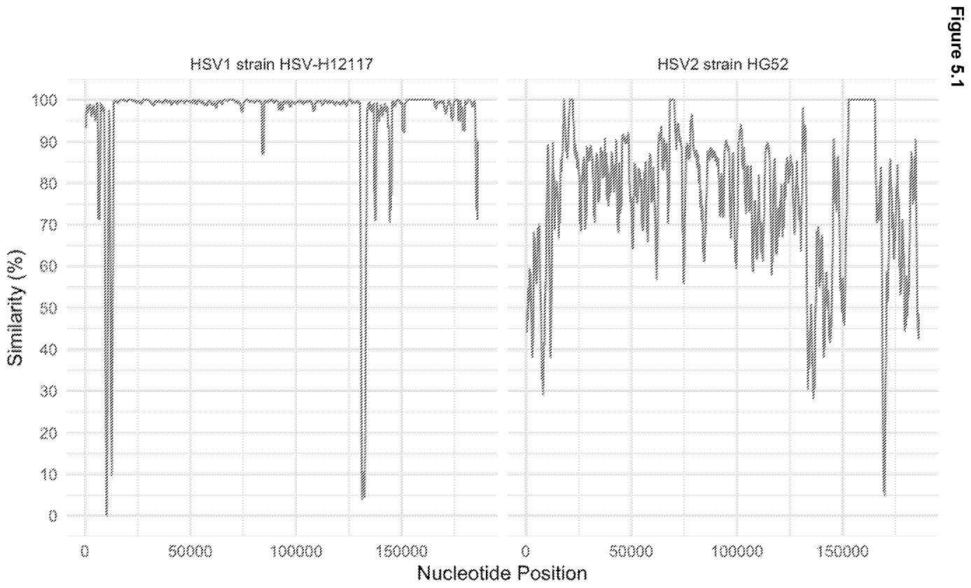

Viral output from up to 5 serial passaging of AdB/C/D libraries in HCT116 and HT29 cells were prepared similarly to Stepwise Diversification Stage 1 methods. Sequencing- and bioinformatics-led viral recombinant analysis was performed to analyse new recombinants and the percentage of virus reads demonstrating recombination events. FIG. 4.1 and FIG. 4.2 demonstrates significantly more virus recombinants being produced from HT29 cells for AdD species, whilst A549 cells produce more AdB and AdC recombinants than HCT116 cells. This data highlights the importance of incorporating multiple cell types as part of the virus diversification process, as different Ad species will have a preferred cell line and recombination rates appear to correlate with rates of virus genome amplification.

### Example 5: Application of the Stepwise Diversification Method to Other Double Stranded DNA Viruses

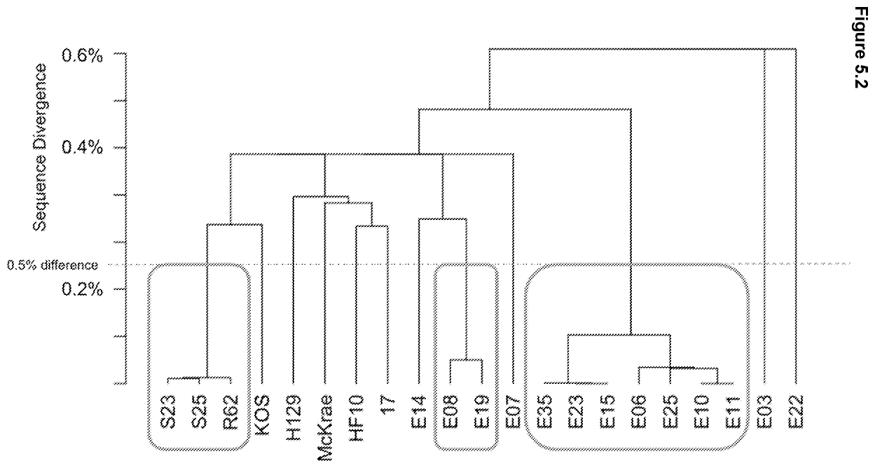

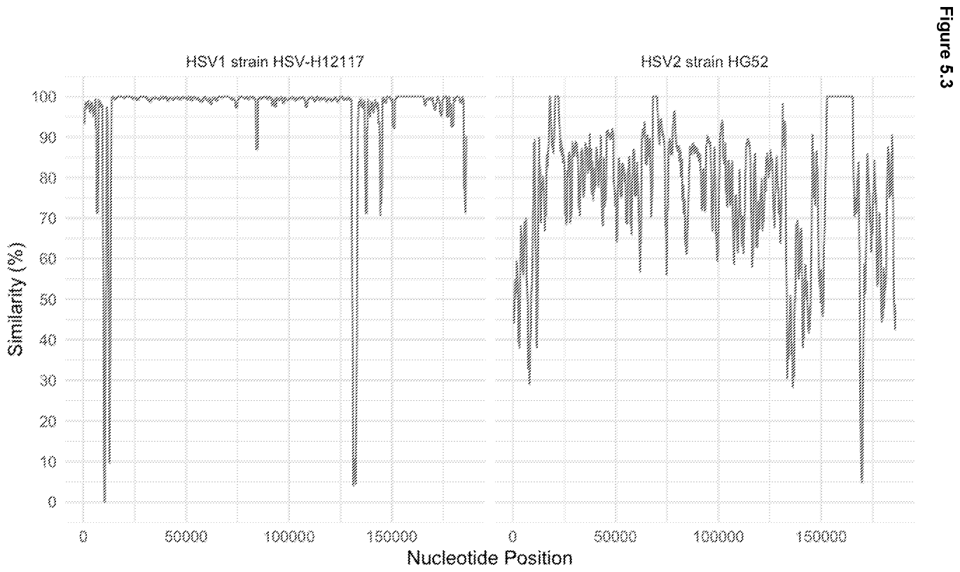

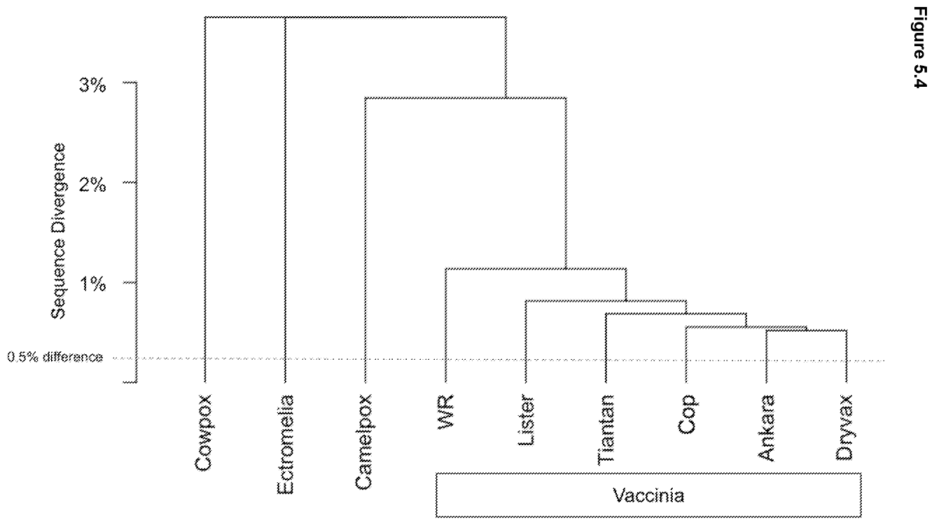

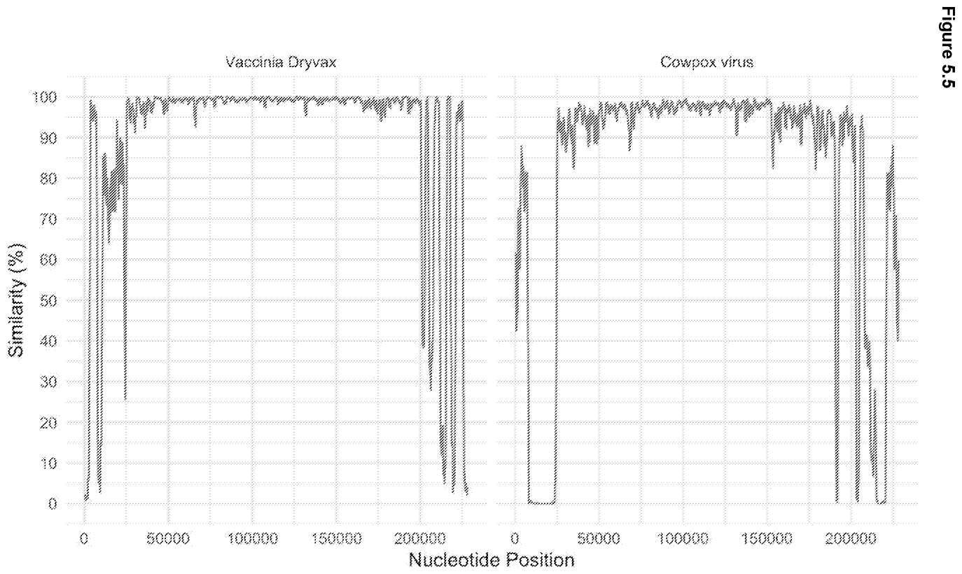

Recombination is observed frequently within adenovirus species, but less commonly between serotypes and species with different infection kinetics and lower levels of homology (FIG. 5.1). FIG. 5.1 shows serotypes in Ad-B1 species to share >98% overall homology with other viruses in AdB-1, 80-90% homology with AdB2, 50-70% with AdC and AdD. Significant levels of homologous recombination is observed within species, including AdB1 and AdB2, but less so between species, indicating that sequence similarity >80% is advantageous for recombinant adenoviruses to be efficiently produced. Hence the process described above (Step-wise Diversification), co-infecting cells with at least two viruses from the same species in their preferred cell line to maximise recombination events prior to combining with more genetically distinct viruses and different species, creates more virus diversity than prior art methods.

Other double stranded DNA viruses are also reported to recombine via co-infection and homologous recombination events (Ricordel et al., “Vaccinia Virus Shuffling: deVV5, a Novel Chimeric Poxvirus with Improved Oncolytic Potency”, 2018, Cancers (Basel); 10(7):231). By combining such viruses in a similar stepwise fashion to adenoviruses, i.e. initially recombining at least two viruses from the same species in their preferred cell lines, prior to combining with viruses from other species, the number and diversity of recombinant viruses are increased. Similarly to adenoviruses, Herpes Simplex Viruses (HSV) or Vaccinia Viruses (VV) of the same species share large stretches of homologous DNA regions (FIGS. 5.2 and 5.3) and similar infection kinetics and tropisms.

Different HSV and VV species become more divergent at the DNA level (FIGS. 5.2 and 5.4), with less shared sequence homology and differing cellular tropisms or infection kinetics (Gerber et al., Differences in the Role of Glycoprotein C of HSV-1 and HSV-2 in Viral Binding May Contribute to Serotype Differences in Cell Tropism”, Virology, 214, 29-39 (1995); Herold et al., “Differences in the susceptibility of herpes simplex virus types 1 and 2 to modified heparin compounds suggest serotype differences in viral entry”, Journal of Virology, Vol. 70, No. 6, 1996; McClain et al., “Cell-Specific Kinetics and Efficiency of Herpes Simplex Virus Type 1 Entry Are Determined by Two Distinct Phases of Attachment”, Virology, Volume 198, Issue 2, 1 Feb. 1994, Pages 690-702; Gates et al., “Development of a High-Content Orthopoxvirus Infectivity and Neutralization Assays”, PLoS ONE 10(10): e0138836, 2015). Consequently, HSV and VV serotypes/strains from the same species are much more likely to recombine, and therefore a Stepwise Diversification process, co-infecting viruses from the same species on their preferred cell line prior to combining with other species, will increase the opportunity for recombination events and overall virus diversity, enabling more virus types to participate in recombination and enhancing the diversity of virus libraries.

This stepwise diversification process is applied to generate diverse libraries of HSV and VV. The resulting diverse HSV and VV libraries is used to identify therapeutic agents for cancer, vaccine or gene therapy applications.

Herpes Simplex Virus

DNA sequence similarity within the HSV1 species is high (FIG. 5.2), with significant stretches of DNA homology (FIG. 5.3 comparing HSV1 strain 17 to H12), indicating ample opportunity for recombination events to occur, similarly to those observed within adenovirus species in the Examples 1-4. FIGS. 5.2 and 5.3 demonstrate sequence similarity between distinct HSV species is significantly lower than within species, suggesting fewer opportunities for recombination to occur.

Stepwise Viral Diversification with HSV

Wldtype HSV strains from HSV-1 and HSV-2 species are obtained from the ATCC or other commercial suppliers, and single viral plaques are purified and propagated as described previously (e.g. by Grosche et al., Herpes Simplex Virus Type 1 Propagation, Titration and Single-step Growth Curves, Bio Protoc. 2019 Dec. 5; 9(23): e3441.), Each single isolate is verified by whole genome sequencing and correct alignment to the corresponding Genbank ID entry.

Stage 1—To Promote More Intra-Species Recombination Events

Viral libraries of HSV-1 strains (including KOS, E06, F, H129, McKrae, HF10 name HSV1 strains; FIG. 5.2) or HSV2 strains (including Seattle, HG52, 186, UL39, UL29) are passaged independently on immortalised cell lines (BHK (baby hamster kidney), VERO cells (African green monkey kidney), HeLa (human cervical cancer) or preferred cell line) in culture media with 10% FCS. Cells are seeded 24 hours prior to infection to achieve confluency of 70 to 90% on inoculation. Cells are inoculated with the HSV-1 strain library or the HSV-2 strain library independently at high MOI in RPM11640 with 20 mM HEPES for 1 hr at room temperature before replacing culture medium and incubating at 37° C. 5% CO₂. Viruses are harvested upon signs of CPE. Following one freeze-thaw cycle, clarified supernatants from the first round of virus infection were added to a sub-confluent layer of cells in T75 flasks in culture media; again each HSV species is passaged independently. The volume of supernatant chosen is that which produced signs of CPE in the following round of infection between <2-5 days.

This cycle of infection on T75 flasks is repeated up to 5 times to introduce recombination events within the HSV species. The output of the final round of infection is deemed the output ‘diversified library’ from Stage 1.

Stage 2—To Provide Opportunity for Novel Intra-Species and Inter-Species Recombination Events

HSV-1 output diversified libraries from Stage 1 is pooled with wild-type HSV-1 strains, a library of HSV-2 strains, and/or the HSV-2 output diversified libraries from stage 1 and passaged together on BHK-21 cells, VERO cells, HELA cells and preferred cell lines. Cells are seeded 24 hours prior to infection to achieve confluency of 70 to 95% on inoculation. Cells are inoculated with the HSV-1 and HSV-2 pooled libraries at high MOI in RPM11640 with 20 mM HEPES for 1 hr at room temperature before replacing culture medium and incubating at 37° C. 5% CO₂. Viruses are harvested upon CPE. Following one freeze-thaw cycle, clarified supernatants from the first round of virus infection are added to a sub-confluent layer of cells in T75 flasks in culture media. The volume of supernatant chosen is that which produces CPE (CPE) in the following round of infection between 2-5 days. This cycle of infection on T75 flasks is repeated up to 5 times to introduce recombination events within the HSV species.

Diversity of a virus library with respect to virus recombination is determined by high throughput next-generation sequencing (NGS) of the virus genomes in that library. Sequences are aligned against a reference set comprising sequences for each known WT virus. Reads mapping to multiple references are confirmed as chimeric using BLAST searches.

Vaccinia Virus

DNA sequence similarity within Vaccinia species is high (FIG. 5.4), with significant stretches of DNA homology (FIG. 5.5 comparing Vaccinia Western Reserve to Dryvax), indicating ample opportunity for recombination events to occur, similarly to those observed within adenovirus species in the Examples above. Sequence similarity between distinct Orthopoxvirus species is lower, suggesting less efficient recombination events occurring.

Stepwise Viral Diversification with Vaccinia Virus

Orthopoxvirus, including vaccinia strains, are obtained from the ATCC, and single viral plaques are purified and propagated as described (e.g. in Cotter et al., “Preparation of Cell Cultures and Vaccinia Virus Stocks”, Curr. Protoc. Microbiol. 2015 Nov. 3; 39: 14A.3.1-14A.3.18 3). Each isolate is verified by Whole Genome Sequencing and correct alignment to the corresponding Genbank ID entry.

Stage 1—To Promote More Intra-Species Recombination Events

Viral libraries of Vaccinia strains (FIG. 5.4) or strains from other Orthopoxvirus species (FIGS. 5.4 and 5.5.) are passaged independently on multiple immortalised cell lines (e.g. BS-C-1 cells, HeLa cells, LoVo cells or preferred cell line) in culture media with 10% FCS. Cells are inoculated with Vaccinia strain or other Orthopoxvirus libraries at high MOI in culture medium with 2.5% FBS for 2 hrs at 37° C. 5% CO₂. Upon signs of CPE, virus is harvested by three freeze thaw cycles to lyse cells. Virus harvested from the first round of infection is sonicated on ice and used to inoculate HeLa cells in a second round of infection at an MOI that would produce signs of CPE in ˜3 days. The volume of supernatant chosen is that which produces CPE in the following round of infection in <3 days. This cycle of infection in culture flasks is repeated up to 5 times to introduce recombination events within the Vaccinia and other Orthopoxvirus species.

Stage 2—To Provide Opportunity for Novel Intra-Species and Inter-Species Recombination Events

Vaccinia output diversified libraries from Stage 1 is pooled with wild-type vaccinia strains, a library of other Orthopoxvirus strains, and/or the Orthopoxvirus output diversified libraries from stage 1 at equal genomes and passaged together on multiple cell lines.

Upon signs of CPE, virus is harvested by three freeze thaw cycles to lyse cells. Virus harvested from the first round of infection is sonicated on ice and used to inoculate cells in a second round of infection at an MOI that would produce CPE in ˜3 days. The volume of supernatant chosen is that which produces signs of CPE in the following round of infection between <3 days.

This cycle of infection in culture flasks is repeated up to 5 times to introduce recombination events within and between Vaccinia and other Orthopoxvirus species.

Diversity of a virus library with respect to virus recombination is determined by high throughput next-generation sequencing (NGS) of the virus genomes in that library. Sequences are aligned against a reference set comprising sequences for each known WT virus. Reads mapping to multiple references are confirmed as chimeric using BLAST searches.

### SEQUENCE LISTING FREE TEXT

<210> 1
<223> AdB Forward Primer
<210> 2
<223> AdB Reverse Primer
<210> 3
20<223> AdC Forward Primer
<210> 4
<223> AdC Reverse Primer
<210> 5
<223> AdD Forward Primer
<210> 6
<223> AdD Reverse Primer

1. A process for producing a library of viruses, the process comprising:
(a) a first culturing step, comprising culturing together, on one or more cell lines, viruses of at least two different serotypes from a first species of double-stranded DNA virus, and
(b) combining
(i) viruses obtained from Step (a), with
(ii) viruses of at least two different serotypes of the same species from each of one or more further species of double-stranded DNA viruses,
wherein the first species of double-stranded DNA virus and each further species of double-stranded DNA virus are all different species in the same family or same genus of double-stranded DNA viruses;
to produce a library of viruses;
and optionally
(c) a second culturing step, wherein the viruses which are combined in Step (b) are cultured together on one or more cell lines; and
(d) combining viruses or portions thereof obtained after Step (c), and/or isolating a plurality of viruses therefrom,
to produce a library of viruses.

2. A process as claimed in claim 1, wherein in Step (a), the viruses of at least two different serotypes from a first species of double-stranded DNA virus are cultured together:
(i) on a single cell line;
(ii) on a plurality of cell lines, wherein the plurality of cell lines are cultured separately; or
(iii) on a plurality of cell lines, wherein the plurality of cell lines are cultured together.

3. A process as claimed in claim 1, wherein, in Step (b)(ii), for each species of the one or more further species of double-stranded DNA virus, the viruses of different serotypes from that species are ones that have previously been cultured together, wherein viruses of different species were previously cultured independently.

4. A process as claimed in claim 1, wherein Steps (a) and (b) comprise:
(a) a first culturing step, comprising
(i) culturing together, on one or more cell lines, viruses of at least two different serotypes from a first species of double-stranded DNA virus; and
(ii) culturing, on one or more cell lines, viruses of at least two different serotypes of the same species from each of one or more further species of double-stranded DNA viruses, wherein, for each species of double-stranded DNA virus, viruses of different serotypes of the same species are cultured together, and viruses of different species are cultured independently;
and
(b) combining
(i) viruses from Step (a)(i), and
(ii) viruses from Step (a)(ii).

5. A process as claimed in claim 1, wherein:
Step (b) additionally comprises combining viruses from (i) and (ii) with viruses from:
(iii) the first species of double-stranded DNA virus;
(iv) one or more wild-type viruses of the same family, genus or species as the first species of double-stranded DNA virus;
(v) one or more of the further species of double-stranded DNA viruses; and/or
(vi) one or more wild-type viruses of the same family, genus or species as one of the further species of double-stranded DNA virus.

6. A process as claimed in claim 1, wherein in Step (c), the viruses of the first species and each further species are cultured together:
(i) on a single cell line;
(ii) on a plurality of cell lines, wherein the plurality of cell lines are cultured separately; or
(iii) on a plurality of cell lines, wherein the plurality of cell lines are cultured together.

7. A process as claimed in claim 1, wherein Step (d) additionally comprises combining viruses or portions thereof obtained after Step (c) with viruses from:
(i) the first species of double-stranded DNA virus;
(ii) one or more of the further species of double-stranded DNA viruses;
(iii) one or more viruses obtained after culturing Step (a);
(iv) one or more wild-type viruses from the same family, genus or species as the first species of double-stranded DNA virus; and/or
(v) one or more wild-type viruses from the same family, genus or species as one of the further species of double-stranded DNA viruses.

8. A process as claimed in claim 1, wherein the viruses are subjected to mutagenesis before, during or after one or more of Steps (a), (b) and/or (c).

9. A process as claimed in claim 1, wherein the double-stranded DNA virus is selected from Adenoviridae, Herpesviridae, and Poxviridae families.

10. A process as claimed in claim 9, wherein the Adenoviridae are species of human adenovirus selected from the group consisting of AdB, AdC, AdD, AdE, AdF and AdG.

11. A process as claimed in claim 10, wherein;
(i) a species is AdB and the serotypes are selected from the group consisting of Ad3, Ad7, Ad11, Ad14, Ad16, Ad21, Ad34, Ad35, Ad50 and Ad55;
(ii) a species is AdC and the serotypes are selected from the group consisting of Ad1, Ad2, Ad5, Ad6 and Ad57;
(iii) a species is AdD and the serotypes are selected from the group consisting of Ad8, Ad9, Ad10, Ad13, Ad15, Ad 17, Ad19, Ad20, Ad22, Ad23, Ad24, Ad25, Ad26, Ad27, Ad28, Ad29, Ad30, Ad32, Ad33, Ad36, Ad37, Ad38, Ad39, Ad42, Ad43, Ad44, Ad45, Ad46, Ad47, Ad48, Ad49, Ad51, Ad53, Ad54 and Ad56; and/or
(iv) a species is AdF and the serotypes are selected from the group consisting of Ad40 and Ad41.

12. A process as claimed in claim 1, wherein the family of double-stranded DNA virus is selected from Herpesviridae and Poxviridae.

13. A process as claimed in claim 1, wherein at least 3, 4, 5, 6, 7, 8, 9, 10 or more different serotypes from a first species are cultured together in Step (a); and/or at least 3, 4, 5, 6, 7, 8, 9, 10 or more different serotypes from one or more further species are combined in Step (b).

14. A process as claimed in claim 1, wherein the number of the one or more further species of double-stranded DNA viruses is 1, 2, 3, 4, 5, 6, 7, 8, 9, 10 or more further species.

15. A process as claimed in claim 1, wherein the one or more cell lines are cancer cell lines, or are selected from the group consisting of A549, HT29, HEK293, HCT116, MM1S, SKOV3, MMR, JJN3, RPMI-8226 and U266 cell lines.

16. A process as claimed in claim 15, wherein:
(i) a species is AdB and the cell line is A549 or HCT116;
(ii) a species is AdC and the cell line is MM1S, HEK293 or A549; and/or
(iii) a species is AdD and the cell line is HT29 or A549.

17. A process as claimed in claim 1, wherein the viruses are passaged 4-6 times, each after 3-7 days, in the first and/or second culturing step.

18. A process as claimed in claim 1, wherein the viruses are passaged 2-6 times, each after 2-6 days, in the first and/or second culturing step.

19. A library which is obtained by or obtainable by a process as claimed in claim 1.

20. A chimeric virus, or a chimeric adenovirus, obtained by or obtainable by a process as claimed in claim 1.


SEQUENCE LISTING (6 sequences)

SEQ ID NO: 1 - DNA, 24 bp, Artificial Sequence
  AdB Forward Primer
gagttggctttaagtttaatgagc

SEQ ID NO: 2 - DNA, 20 bp, Artificial Sequence
  AdB Reverse Primer
tgaggcctgataaacagtat

SEQ ID NO: 3 - DNA, 20 bp, Artificial Sequence
  AdC Forward Primer
gcttaatgaccagacaccgt

SEQ ID NO: 4 - DNA, 19 bp, Artificial Sequence
  AdC Reverse Primer
ggtatatgcaaaggtggca

SEQ ID NO: 5 - DNA, 18 bp, Artificial Sequence
  AdD Forward Primer
gggatgatgaccgagctg

SEQ ID NO: 6 - DNA, 18 bp, Artificial Sequence
  AdD Reverse Primer
cagacatgcctgctacat